(19) United States
(12) Patent Application Publication
(10) Pub. No.: US 2008/0254130 A1
(43) Pub. Date: Oct. 16, 2008

(54) Skin Antiaging & Brightening via Multi-function Treatment of Enzyme Dysfunction

(75) Inventor: SHYAM K. GUPTA, SCOTTSDALE, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, SCOTTSDALE, AZ (US)

(21) Appl. No.: 12/139,462

(22) Filed: Jun. 14, 2008

Related U.S. Application Data
(63) Continuation-in-part of application No. 10/604,999, filed on Aug. 29, 2003, now Pat. No. 7,320,797.

(51) Int. Cl. A61K 9/14; A61K 31/12; A61K 31/47; A61K 31/435; A61K 31/428; A61K 31/44; A61K 31/40; A61K 31/4164; A61P 17/00; A61K 31/426; A61K 31/505; A61K 31/404; A61K 31/5415; A61K 31/4965; A61K 31/517; A61K 8/18

(52) U.S. Cl. 424/489; 514/689; 514/312; 514/297; 514/367; 514/354; 514/423; 514/399; 514/365; 514/256; 514/419; 514/226.2; 514/255.06; 514/266.1; 424/49

(57) ABSTRACT

The present invention relates to a topical method of treatment for dysfunction of certain dermal enzymes, and the treatment of skin condition or disorder caused by said dysfunction. The said method of treatment consists of (i) an extra-cellular, matrix metalloprotease regulating agent, and (ii) an intra-cellular ubiquitin—proteasome regulating agent, and (iii) an epidermal melanocyte-regulating agent; and, wherein, said extra-cellular agent, said intracellular agent, and said epidermal agent can, surprisingly and unexpectedly, be a single multi-function compound having chemical formula (I). Additionally, the method of the present invention provides treatment of skin condition or disorder caused by dysfunction of said dermal enzymes; wherein said skin disorder is skin aging, skin wrinkles, dark skin, age spots, acne, skin inflammation, loss of cellular antioxidants, loss of collagen, loss of skin pliability, loss of skin suppleness, oily skin, or a combination thereof:

The present invention is a continuation-in-part of U.S. patent application Ser. No. 10/604,999 (filed on Aug. 29, 2003), now U.S. Pat. No. 7,320,797.

### BACKGROUND OF THE INVENTION

The present invention relates to a topical method of treatment for dysfunction of certain dermal enzymes, and the treatment of skin disorders caused by said dysfunction. The said method of treatment consists of (i) an extra-cellular, matrix metalloprotease regulating agent, and (ii) an intra-cellular ubiquitin—proteasome regulating agent, and (iii) an epidermal melanocyte-regulating agent; and, wherein, (iv) said extra-cellular agent, said intracellular agent, and said epidermal agent can, surprisingly and unexpectedly, be a single multi-function compound; “multi-function” having been defined herein as a compound that can perform multiple biological functions concurrently, or allows for an unlimited number of functions, each of these functions operates independently of the others. Additionally, the method of the present invention provides treatment of skin condition or disorder caused by dysfunction of said dermal enzymes; wherein said skin disorder can be, among others, skin aging, skin wrinkles, dark skin, age spots, acne, skin inflammation, loss of cellular antioxidants, loss of collagen, loss of skin pliability, loss of skin suppleness, oily skin, or a combination thereof.

### DESCRIPTION OF THE RELATED ART

The enhancement of physical appearance occupies greater focus in human life than nearly all other daily life-related concerns combined. There are far more consumer products available for the beautification of human body than for the treatment of human ailments. The improvement of skin tone and appearance is a growing, multibillion-dollar industry encompassing cosmetic, nutraceutical, pharmaceutical, and physical therapy disciplines. The consumer attention is focused on newest miracle ingredient in age-defying, anti-wrinkle, skin smoothing, skin brightening, and other similar antiaging compositions, for example, U.S. Patent Application 20030091665 (Lu et al.), 20030083380 (Yu et al.), 20020048798 (Avery et al.), 20020034527 (Streicher et al.), 20030091666 (Murad), 20030157138 (Eini et al.), U.S. Pat. No. 6,514,507 (Maignan et al.), U.S. Pat. No. 6,284,233 (Simon et al.), U.S. Pat. No. 6,436,416 (Grainger et al.), U.S. Pat. No. 6,224,850 (Breton et al.), U.S. Pat. No. 6,569,683 (Ochi), and U.S. Pat. No. 5,885,596 (Parab).

Oligopeptides have received much attention in recent prior art for treating topical condition of skin aging. Bakala et al. (EP 1,786,386) disclose cosmetic use of at least one type of natural tetrapeptide or one of the analogs thereof in the form of a skin antiaging and restructuring agent. Argireline is another highly commercialized oligopeptide for skin disorder including wrinkles.

Dong et al. (WO 2008054144) disclose certain cathespin G inhibitors for preventing skin aging.

Pierfrancesco (WP 2007099172) discloses the use of melatonin associated with immunoactive and antioxidant substances significantly potentiating its antiaging activity, remarkably reducing free radical formation and thereby improving the aspect of both skin and hair.

Moon Hee (WO 2007075016) discloses a collagenase inhibitor containing a poly-gamma-glutamic acid-vitamin C complex and a composition for preventing skin wrinkles, which contains said conjugate and can be used in drugs, cosmetics and foods. This conjugate has not only the effect of inhibiting collagenase activity, but also antiaging effects, such as an antioxidant effect and a skin wrinkle-improving effect. Also, the collagenase inhibitor maintains the elasticity of the skin by keeping skin connective tissue taut and has high skin compatibility, excellent moisturization effect, moisture-absorbing effect and sustained-release effect.

Soo et al. (KR 20050022251) disclose a hydroxamic acid derivative that shows an antiaging effect and is improved in safety and stability to the skin stimulus and discoloration, its preparation method, and an antiaging skin preparation for external use, and a collagenase and elastase expression inhibitor.

Katsuyoshi et al. (JP 2006225286) disclose extract of Punica granatum flowers to be very high in antiaging, radical scavenging, skin brightening, and collagenase activity-inhibitory effects. Various skin care preparations for the above applications can be obtained. Ellagic acid, obtained from pomegranate fruit extract, is also well known for its very similar skin beneficial activity.

Mahalingham et al. (TW 280139B) disclose a method and composition that comprises (a) a de-pigmenting agent or anti-aging agent in an amount effective to prevent, treat and/or ameliorate pigmentation or the various signs of aging at an area of skin to which it is applied, and (b) a cosmetically or pharmaceutically acceptable vehicle. Suitable de-pigmenting agents include 3,3′-thiodipropionic acid, thiazolidine-2-carboxylic acid, Kaempferol-7-glucoside, perilla oil, and clofibrate and clofibrate analogs and/or derivatives, as well as those set forth below. Suitable antiaging agents include 3,3′-thiodipropionic acid and/or its derivatives. Lipoic acid, having a related chemical structure, is also well known for its antioxidant and wrinkles reduction properties.

Rika et al. (WO 2008001465) disclose a moisturizer, an antiaging agent, a skin whitening agent and an antioxidant, which contain an extract of Piper betel as an active ingredient.

Yoshizaki et al. (JP 2008031008) disclose certain MMP inhibitors for skin antiaging.

Gupta (U.S. patent application Ser. No. 20060074108) discloses certain MMP inhibitors and their application in the treatment of topical disorder.

Gupta (U.S. patent application Ser. No. 20060269494) discloses certain ubiquitin—proteasome modulators and their application in the treatment of topical disorder.

Gupta (U.S. patent application Ser. No. 20050271608) discloses certain tyrosinase inhibitors for skin whitening.

However, none of the methods reported in the prior art, some of which are referenced above, provide a comprehensive, one step solution to the problems of skin aging and skin condition caused by dysfunction of a combination of certain key dermal enzymes. Moreover, none of the methods of the prior art provide a concurrent treatment for the dysfunction of said combination of dermal enzymes.

Based on the science of skin biology, the following three aspects are incorporated in the method of the present invention to provide a comprehensive solution to the problems associated with skin disorder caused by the dysfunction of certain dermal enzymes: (i) an extra-cellular, matrix metalloprotease regulating agent, and (ii) an intra-cellular ubiquitin—proteasome regulating agent, and (iii) an epidermal melanocyte-regulating agent. This in itself is unprecedented in the prior art; the fact that a single multi-function compound can achieve all of the above three feats concurrently is even more unprecedented and surprising.

### BRIEF SUMMARY OF THE INVENTION

The dysfunction of key dermal enzymes, matrix metalloproteases (MMP), which are extra-cellular enzymes; ubiquitin—proteasome system, which is an intra-cellular system of enzymes; and melanocyte regulating enzymes, which are epidermal enzymes, causes a number of topical disorders, which includes skin aging, skin wrinkles, dark skin, age spots, acne, skin inflammation, loss of cellular antioxidants, loss of collagen, loss of skin pliability, loss of skin suppleness, oily skin, or a combination thereof.

There is a need for a method that can provide a multi-function treatment for the dysfunction of MMP, ubiquitin—proteasome system, and melanocyte regulating enzymes. Said method would provide novel cosmetic and pharmaceutical treatments for skin ailments that include skin aging, skin wrinkles, dark skin, age spots, acne, skin inflammation, wound, loss of cellular antioxidants, loss of collagen, loss of skin pliability, loss of skin suppleness, oily skin, or a combination thereof.

The present method relates to a multi-function comprehensive treatment of problems of topical condition or disorder associated with dysfunction of three key dermal enzymes: (i) extra-cellular matrix metalloproteases, and (ii) intra-cellular ubiquitin—proteasomes, and (iii) epidermal melanocyte-regulating enzymes, and, wherein, (iv) said extra-cellular agent, said intracellular agent, and said epidermal agent can, surprisingly and unexpectedly, be a single multi-function compound.

### BRIEF DESCRIPTION OF THE SEVERAL VIEWS OF THE DRAWINGS

FIG. 1 Multi-function Compound.

FIG. 2 Hydroxyaryl Compound with Additional Rings.

FIG. 3 N-Heterocyclic Multi-function Compounds.

FIG. 4 2-Acetyl Substituted N-Heterocyclic Multi-function Compounds.

FIG. 5 N-Heterocyclic Multi-function Compounds with Additional Cyclic Rings.

FIG. 6 N-Heterocyclic Multi-function Compounds with Additional Heteroatoms.

### DETAILED DESCRIPTION

A topical method of treatment for a comprehensive solution to the problems associated with skin aging requires the following: (i) treatment of the dysfunction of extra-cellular, matrix metalloproteases, and (ii) treatment of the dysfunction of intra-cellular ubiquitin—proteasomes, and —(iii) treatment of the dysfunction of epidermal melanocyte-regulating enzymes. Most preferably, it would also be advantageous if a single compound or agent can provide all of the above functions concurrently.

Gupta (U.S. patent application Ser. No. 20060074108) discloses certain MMP inhibitors and their application in the treatment of topical disorder.

Gupta (U.S. patent application Ser. No. 20060269494) discloses certain ubiquitin—proteasome modulators and their application in the treatment of topical disorder.

Gupta (U.S. patent application Ser. No. 20050271608) discloses certain epidermal melanocyte-regulating tyrosinase inhibitors for skin whitening.

However, Gupta does not teach a multi-function treatment that can provide all of the above benefits in a single treatment or methodology.

The Dysfunction of MMP.

Matrix metalloproteases are naturally occurring proteases found in most mammals and are zinc-dependent endopeptidases that perform extracellular tissue reorganization (matrix reorganization).

Proteases catalyze amide (peptide) bond hydrolysis in protein or peptide substrates. Proteases are classified by (a) their site of action, such as exopeptidases and endopeptidases, or (b) by their reaction mechanisms and nature of active-site residues involved in such mechanisms, such as serine proteases, cysteine proteases, aspartyl proteases, and zinc proteases (also called metalloproteases). The serine and cysteine proteases act directly as nucleophiles to attack the substrate. The aspartyl and zinc proteases activate water molecules as the direct attacking species on the peptide bond. For example, in case of zinc proteases (zinc MMP) one atom of Zn++ is coordinated to two histidine and one glutamic acid side chains in the active-site. One water molecule binds with activated Zn site to form Zn—OH, which is then ready to attack the substrate peptide bond. Once the substrate protein is bound to the active site, zinc can coordinate to the carbonyl oxygen of the peptide bond to be attacked, lowering barriers electronically for [HO—] attack. A conserved glutamate side chain in the active site now acts as catalytic base to protonate the amine product as it leaves the site.

One major biological function of the matrix metalloprotease (MMP) is to catalyze the breakdown of connective tissue or extracellular matrix by virtue of their ability to hydrolyze various components of the tissue or matrix. Examples of the components that may be hydrolyzed by an MMP include collagens (for example, Collagenases type I, II, III, or IV), gelatins (for example, Gelatinases), proteoglycans, and fibronectins. Apart from their role in degrading connective tissue, MMP are also involved in the activation of the zymogen (pro) forms of other MMP thereby inducing MMP activation (proenzyme activation). They are also involved in the biosynthesis of TNF-alpha which is implicated in many pathological conditions and can cause or contribute to the effects of inflammation, rheumatoid arthritis, asthma, autoimmune disease, multiple sclerosis, graft rejection, fibrotic disease, cancer, infectious diseases, malaria, mycobacterial infection, meningitis, fever, psoriasis, cardiovascular/pulmonary effects (e.g., post-ischemic reperfusion injury), congestive heart failure, hemorrhage, coagulation, hyperoxic alveolar injury, radiation damage, cachexia, anorexia, and acute phase responses like those seen with infections and sepsis and during shock (e.g., septic shock and hemodynamic shock). Recent reviews of MMP are presented by Albrecht Messerschmidt, Wolfram Bode, and Mirek Cygler (Editors), (2004) Handbook of Metalloproteins, Volume 3, John Wiley, NY; Ivano Bertini, Astrid Sigel, and Helmut Sigel (Editors), (2001) Handbook on Metalloproteins, Marcel Dekker, NY; Woessner and Nagase, (2000) “Matrix metalloproteases and TIMPs”, Oxford University Press, Oxford; and Doherty et al. (2002) Expert Opinion Therapeutic Patents 12(5): 665-707.

Over 30 MMP have been characterized so far in humans and several major groups have been determined based on substrate specificity, some of which are described below, and are believed applicable to the present invention.

MMP-1 (also known as collagenase 1, or fibroblast collagenase). The substrates of MMP-1 include collagen I, collagen II, collagen III, gelatin, and proteoglycans. Over-expression of this enzyme is believed to be associated with emphysema, with hyperkeratosis and atherosclerosis, overexpressed alone in papillary carcinoma.

MMP-2 (also known as gelatinase A, basement membrane collagenase, or proteoglycanase). The substrates of MMP-2 include collagen I, collagen II, collagen IV, collagen V, collagen VII, collagen X, collagen Xi, collagen XIV, elastin, fibronectin, gelatin, nidogen, believed to be associated with tumor progression through specificity for type IV collagen (high expression observed in solid tumors and believed to be associated with their ability to grow, invade, develop new blood vessels and metastasize) and to be involved in acute lung inflammation and in respiratory distress syndrome.

MMP-3 (also known as stromelysin 1). The substrates of MMP-3 include collagen III, collagen IV, collagen V, collagen IX, collagen X, laminin, nidogen, overexpression believed to be involved in atherosclerosis, aneurysm and restenosis.

MMP-7 (also known as matrilysin). The substrates of MMP-7 include collagen IV, elastin, fibronectin, gelatin, laminin.

MMP-8 (also known as collagenase 2, or neutrophil collagenase). The substrates of MMP-8 include collagen I, collagen II, collagen III, collagen V, collagen VII, collagen IX, gelatin over-expression of which can lead to non-healing chronic ulcers.

MMP-9 (also known as gelatinase B, or 92 kDa gelatinase). The substrates of MMP-9 include collagen I, collagen III, collagen IV, collagen V, collagen VII, collagen X, collagen XIV, elastin, fibronectin, gelatin, nidogen The above enzyme is believed to be associated with tumor progression through specificity for type IV collagen, to be released by eosinophils in response to exogenous factors such as air pollutants, allergens and viruses, to be involved in the inflammatory response in asthma and to be involved in acute lung inflammation and respiratory distress syndrome. The applicants believe that an inhibitor for this enzyme would be effective in the treatment of chronic obstructive pulmonary disorder (COPD) and/or asthma.

MMP-10 (also known as stromelysin 2). The substrates of MMP-10 include collagen III, collagen IV, collagen V, elastin, fibronectin, and gelatin.

MMP-11 (also known as stromelysin 3). The substrates of MMP-11 include serine protease inhibitors (Serpins).

MMP-12 (also known as metalloelastase, human macrophage elastase, or HME). The substrates of MMP-12 include fibronectin, laminin, believed to play a role in tumor growth inhibition and regulation of inflammation and to play a pathological role in emphysema and in atherosclerosis, aneurysm and restenosis. The applicants believe that an inhibitor for this enzyme would be effective in the treatment of COPD and/or asthma.

MMP-13 (also known as collagenase 3). The substrates of MMP-13 include collagen I, collagen II, collagen III, collagen IV, collagen IX, collagen X, collagen XIV, fibronectin, and gelatin, recently identified as being overexpressed alone in breast carcinoma. The applicants believe that an inhibitor for this enzyme would be effective in the treatment of breast cancer and arthritis.

MMP-14 (also known as membrane MMP or MT1-MMP). The substrates of MMP-14 include MMP-2, collagen I, collagen II, collagen III, fibronectin, gelatin, laminin.

MMP-15 (also known as MT2-MMP). The substrates of MMP-15 include MMP-2, collagen 1, collagen II, collagen III, fibronectin, laminin nidogen.

MMP-16 (also known as MT3-MMP). The substrates of MMP-16 include MMP-2, collagen 1, collagen III, fibronectin.

MMP-17 (also known as MT4-MMP), substrates fibrin (fibrinogen).

MMP-18 (also known as collagenase 4).

MMP-19 (also known as Rasi-1). The substrates of MMP-19 include MMP-9, gelatin, laminin-1, collagen IV, and fibronectin.

MMP-20 (also known as enamelysin), substrate amelogenin.

MMP-23 (also known as femalysin), substrate gelatin.

MMP-24 (also known as MT5-MMP). The substrates of MMP-24 include MMP-2, gelatin, fibronectin, chondroitin, and dermitin sulfate proteoglycans.

MMP-25 (also known as MT6-MMP). The substrates of MMP-25 include MMP-2, gelatin, collagen IV, and fibronectin.

MMP-26 (also known as matrilysin 2 or endometase). The substrates of MMP-26 include denatured collagen, fibrinogen, fibronectin, vitronectin.

MMP-28; also known as epilysin, substrates caesin.

Dysfunction due to over-activation of a matrix metalloprotease (“MMP”), or an imbalance between an MMP and a natural (i.e., endogenous) tissue inhibitor of a matrix metalloprotease (“TIMP”), has been linked to the pathogenesis of diseases characterized by the breakdown of connective tissue or extracellular matrix. Examples of diseases characterized by over-expression and/or over-activation of an MMP include rheumatoid arthritis, asthma, osteoarthritis; osteoporosis; periodontitis; multiple sclerosis; gingivitis; corneal, epidermal, and gastric ulceration; atherosclerosis; neointimal proliferation, which leads to restenosis and ischemic heart failure; stroke; renal disease; macular degeneration; and tumor metastasis.

Further, some MMP-mediated diseases may involve over activity of only one MMP enzyme. This is supported by the recent discovery that MMP-13 alone is over-expressed in breast carcinoma, while MMP-1 alone is over-expressed in papillary carcinoma.

“MMP-associated disorder” which is treatable according to the present invention encompasses all disorders in which the expression and/or activity of at least one MMP needs to be decreased irrespective of the cause of such disorders. Such disorders include, for example, those caused by inappropriate ECM degradation. Illustrative but not limiting examples of such MMP-associated disorders are: Cancer; Inflammatory disorders such as inflammatory bowel diseases, multiple sclerosis, glomerulonephritis, and uveoretinitis; Lung diseases such as chronic obstructive pulmonary disorder, asthma, acute lung injury, and acute respiratory distress syndrome; Dental diseases such as periodontal disease and gingivitis; Joint and bone diseases such as osteoarthritis and rheumatoid arthritis; Liver diseases such as liver fibrosis, cirrhosis and chronic liver disease; Fibrotic diseases such as pulmonary fibrosis, lupus, glomerulosclerosis, systemic sclerosis and cystic fibrosis; Vascular pathologies such as aortic aneurysm, atherosclerosis, hypertension, cardiomyopathy and myocardial infarction; and Restenosis.

Opthalmologic disorders such as diabetic retinopathy, dry eye syndrome, macula degeneration and corneal ulceration; wound healing disorders such as non healing ulcers, excessive scar formation; Tissue ulceration such as gastric ulcers and skin ulcers; Skin disorders such as psoriasis, acne, rosacea, skin discoloration, and skin aging; Uterus and pregnancy-related disorders such as adenomyosis and pre-eclampsia.

Disorders caused by pathogens such as HIV-1 infection, bacterial meningitis.

Central nervous system disorders such as Alzheimer's disease; Neuroinflammatory disorders such as multiple sclerosis and acute neuroinflammation; and also Marfan syndrome, invertebral disk degeneration, graft-versus-host disease and lupus.

Research has been carried out into the identification of inhibitors for dysfunctional over-active MMP that are selective, for example, for a few of the MMP subtypes. A MMP inhibitor of improved selectivity would avoid potential side effects associated with inhibition of MMP that are not involved in the pathogenesis of the disease being treated. Further, use of more selective MMP inhibitors would require administration of a lower amount of the inhibitor for treatment of disease than would otherwise be required and, after administration, partitioned in vivo between multiple MMP. Still further, the administration of a lower amount of compound would improve the margin of safety between the dose of the inhibitor required for therapeutic activity and the dose of the inhibitor at which toxicity is observed.

Whittaker et al., Chem. Rev., 1999, 99, 2735-2776, reviewed the design and therapeutic application of matrix metalloprotease inhibitors. The authors explained that the requirement for a molecule to be an effective inhibitor of the MMP class of enzymes is a functional group (e.g. carboxylic acid, hydroxamic acid or sulfhydryl) capable of chelating to the active site zinc II ion, at least one functional group that provides a hydrogen bond interaction with the enzyme backbone, and one or more side chains which undergo effective van der Waals interactions with the enzyme sub sites. A large number of such compounds are mentioned in which chelation is by a hydroxamate group.

Chen et al., J. Am. Chem. Soc, 2000, 122, 9648-9654 disclose a potent and selective inhibitor for MMP-13. The authors had found that a compound referenced CL-82198 exhibited weak inhibition of MMP-13 but complete lack of activity against MMP-1 and MMP-9. Chen at al. postulated that the above compound sits in and extends along the S1′ pocket of MMP-13, with the morpholine group forming a hydrogen bond with the backbone amide group of Leu-82 and with the benzofuran group packing deep into the S1′ pocket, but not binding to zinc of the catalytic domain. The authors decided that the way forward in the design of an MMP-13-selective lead compound was to make a compound that had both a moiety that chelates to zinc of the catalytic domain and a moiety that sits in the S1′ pocket, and arrived at a potent compound called WAY-170523 that shows >5800-, 56- and 500-fold selectivity against MMP-1 and MMP-9.

Stallings et al (WO 01/05389) disclose certain N-hydroxy compounds located adjacent to an aryl ring. These compounds have shown strong binding with the catalytic zinc atom in the active-site of MMP.

Further compounds that exhibit selectivity for MMP-12 are described in WO 01/83431 and WO 01/83461 (Shionogi) and are stated to be effective against emphysema and COPD. They rely for activity on the presence of groups that chelate to zinc.

Curtin et al., [Bioorg. Med. Chem. Lett. 11 (2001), 1557-1560] disclose MMP inhibitors bearing a zinc-binding group, which were reported to be highly selective for MMP-2 versus MMP-1.

Wada et al, [J. Med. Chem., 45, (20020, 219-232], discovered a compound that is selective for the inhibition of MMP-2 and MMP-9 over MMP-1, and which demonstrated antitumor activity in a murine syngenetic tumor growth model. These authors attribute selectivity in MMP to differences in the depth of the S1′ pocket and classify the MMP into those with relatively deep pockets (MMP-2, -3, -8, -9, and -13) and those with shallow pockets (MMP-1 and -7). Selectivity is achieved by incorporation of an extended so-called P1′ group such as biphenyl for fitting into the S1′ pocket whereas the incorporation of smaller P1′ groups generally leads to broad-spectrum inhibition. Again, the above compounds achieve activity by the presence of groups that chelate to zinc.

Dublanchet et al. (U.S. Patent Application 20040171543) disclose MMP inhibitors based on certain hydroxamic acid derivatives.

Jarrousse et al. (U.S. Pat. No. 6,645,477) disclose certain MMP and TIMP inhibitors useful for hair growth modulation (i.e. to stimulate hair growth or to retard hair growth).

Wang et al. (U.S. Patent Application 20020037827) disclose the identification of MMP-25 in skin cells and its role in hair growth. The methods for inhibiting MMP-25 activity, leading to the methods useful for inhibiting hair growth are also disclosed.

O'Brien et al. (U.S. Patent Application 20040029945) disclose a method of inhibiting MMP using compounds that are dibenzofuran sulfonamide derivatives. More particularly, O'Brien invention relates to a method of treating diseases in which matrix MMP are involved such as multiple sclerosis, atherosclerotic plaque rupture, restenosis, aortic aneurism, heart failure, periodontal disease, corneal ulceration, burns, decubital ulcers, chronic ulcers or wounds, cancer metastasis, tumor angiogenesis, arthritis, or other autoimmune or inflammatory diseases dependent upon tissue invasion by leukocytes.

Tsuji et al. (U.S. Patent Application 20040175349) report a method of inhibiting hair growth, which comprises administering an inhibitor of elastase-like enzymes or a neutral endopeptidase inhibitor, and use of an inhibitor of elastase-like enzymes or a neutral endopeptidase inhibitor for the preparation of a hair-growth inhibitor.

Newton et al. (U.S. Patent Application 20040176393) provide a method of treating and preventing heart failure and other vascular diseases in a mammal comprising administering an effective amount of a matrix metalloproteinase inhibitor together with a statin. The invention also provides a method for treating and preventing ventricular dilatation comprising administering an effective amount of a MMP inhibitor together with a statin. The MMP inhibitor to be utilized is a substituted bicyclic compound.

Baarlam et al. (U.S. Patent Applications 20040176386 and 20040171641) disclose compounds useful as metalloproteinase inhibitors, especially as inhibitors of MMP 13.

Becker et al. (U.S. Patent Application 20040167182) disclose certain hydroxamic acid and amide compounds (including salts of such compounds), and, more particularly, to aryl- and heteroaryl-arylsulfonylmethyl hydroxamic acids and amides that inhibit protease activity, particularly MMP activity and/or aggrecanase activity.

Klingler et al. (U.S. Patent Application 20040167120) disclose pyrimidine-4,6-dicarboxylic acid diamides that are suitable for selectively inhibiting collagenase (MMP 13). The pyrimidine-4,6-dicarboxylic acid diamides can therefore be used for treating degenerative joint diseases.

VanZandt et al. (U.S. Patent Application 20040127500) disclose certain MMP inhibitor compounds.

Bunker et al. (U.S. Patent Application 20040142950 and 20040044000) discloses compounds that are inhibitors of MMP-13. The compounds are useful for treating diseases mediated by MMP-13, including the diseases recited herein such as breast cancer, cartilage damage, rheumatoid arthritis, and osteoarthritis.

Ott et al. (U.S. Patent Application 20040132693) disclose spiro-cyclic beta-amino acid derivatives, which are useful as MMP, TNF-alpha converting enzyme (TACE), and/or aggrecanase inhibitors.

King et al. (U.S. Patent Application 20040116491) disclose hydantoin derivatives, which are useful as inhibitors of MMP, TNF-alpha converting enzyme (TACE), aggrecanase, or a combination thereof.

Monroe et al. (U.S. Patent Application 20040105897) disclose composition containing one or more of zinc ions, calcium ions, rubidium ions and/or potassium ions in a pharmaceutically acceptable carrier, which, when administered to a patient in need thereof, effectively modulates the activity of at least MMP-2 and/or MMP-9 in the wound. These inventors have identified MMP-2 and MMP-9 in increased quantities in certain medical conditions. In one such medical condition, MMP have been noted to be involved both in the peripheral region and particularly within the deep recesses of a chronic wound. It has also been a noted increase in these MMP in “difficult to heal” open wounds. Further the present inventors have discovered a synthesized composition which, when clinically introduced to a site exhibiting the presence of one or more MMP effectively shuts down the activity of MMP. This therapeutic effect is particularly evident with respect to the modulation of MMP-2 and MMP-9, as evidenced by analysis of wound cultures for the presence of MMP 2 and 9, and resulting visually observable improvement in the healing of the wound.

Hayakawa et al (U.S. Patent Application 20040082630) disclose certain.alpha-amino-N-hydroxy-acetamide derivatives, wherein R is di-lower alkyl amino, 1,2,3-triazol-2-yl or 1,2,4-triazol-4-yl, m represents an integer from 1 up to and including 10, and n represents an integer from 0 up to and including 10, and the use of such hydroxamic acid derivatives as medicaments, and a method of treating conditions or diseases mediated by MMP using said derivatives.

Johnson et al. (U.S. Patent Application 20040063673) disclose pharmaceutical compositions together with a pharmaceutically acceptable carrier that provides methods of inhibiting an MMP-13 enzyme.

Heinicke et al. (U.S. Patent Application 20040044013 and 20040023953) disclose certain dimercaptoalkyl-substituted quinazoline-2,4(1H,3H)diones. Compounds of this substance class show pharmacologically interesting MMP-inhibitory effect.

Gaudilliere et al. (U.S. Patent Application 20040006077) disclose certain thiazine and oxazine derivatives as MMP-13 inhibitors.

Arnold et al. (U.S. Patent Application 20030225272) disclose certain N-[2(R)-Nonylsuccinic acid]-L-tyrosine-N-2-(N-morpholino)ethylamide; N-[2(R)-Nonylsuccinic acid]-L-phenylalanine-N-3-(N-morpholino)propylamide-; N-[2(R)-Nonylsuccinic acid]-L-valine-N-2-(N-morpholino)ethylamide; N-[2(R)-Nonylsuccinic acid]-L-tyrosine-N-(4-methoxyphenyl)amide; N-[2(R)-Nonylsuccinic acid]-L-phenylalanine-N-(4-methoxyphenyl)amide; N-[2(R)-Nonylsuccinic acid]-L-norvaline-N-(4-methoxyphenyl)amide; N-[2(R)-Nonylsuccinic acid]-L-arginine-N-(4-methoxyphenyl)amide; N-[2(R)-Nonylsuccinic acid]-L-phenylglycine-N-methylamide; N-[2(R)-Nonylsuccinic acid]-L-tyrosine-N-cyclopentylamide; and N-[2(R)-Nonylsuccinic acid]-L-tyrosine-N-3-dimethylaminopropylamide [FIG. 20] useful as MMP-2 and MMP-9 inhibitors.

Among other recent prior art disclosures, Li (U.S. Patent Application 200400439830 and 20040038960), Picard (U.S. Patent Application 200400439790), Ortwine (U.S. Patent Application 200400389740), Nahra et al. (U.S. Patent Application 200400389730), Bunker et al. (U.S. Patent Application 20040038961 and 20040038959), Roark (U.S. Patent Application 20040034009), Frescos et al. (U.S. Patent Application 20040024024), and Getman et al. (U.S. Patent Application 20040034071) teach additional methods for inhibiting various MMP for the control of ailments associated with MMP.

Varani et al. (U.S. Patent Application 20040034098) disclose that chronological aging of human skin can be delayed with the topical application of an MMP inhibitor, preferably a retinoid (an indirect MMP inhibitor). Retinoids also normalize procollagen biosynthesis. Chronological aging, or natural aging, is evidenced in elderly (80+ years old) skin by increased MMP levels and decreased procollagen levels when compared with younger individuals. Prophylactic treatment of not yet chronologically-aged skin with a retinoid both inhibits degradation of dermal collagen and restores procollagen synthesis.

Quirk (U.S. Patent Applications 20040127420 and 20030166567) report inhibitors of MMP useful for treating wounds. The inhibitors are peptides having sequences related to cleavage regions of the proenzyme forms of MMP. The peptide inhibitors of the invention can be formulated into therapeutic compositions and wound dressings that facilitate healing.

Additional references for prior art methods for inhibition of dysfunctional MMP and their applications in medicine include: Agren, M. S. (1999). Matrix metalloproteases (MMP) are required for re-epithelialization of cutaneous wounds. Arch. Dermatol. Res. 291, 583-590; Becker, J. W., Marcy, A. I., Rokosz, L. L., Axel, M. G., Burbaum, J. J., Fitzgerald, P. M., Cameron, P. M., Esser, C. K., Hagmann, W. K., Hermes, J. D., and Springer, J. P. (1995). Stromelysin-1: Three dimensional structure of the inhibited catalytic domain and of the C-truncated proenzyme. Protein Sci. 4, 1966-76; Brown, R. L., Breeden, M P., and Greenhalgh, M D., (1994). PDGF and TGF-a act synergistically to improve wound healing in the genetically diabetic mouse. J. Surg. Res. 56, 562-570; Browner, M. F., Smith, W. W., Castelhano, A. L. (1995). Matrilysin-inhibitor complexes: Common themes among 18 metalloproteinases. Biochemistry 34, 6602-10; Di Colandrea, T., Wang, L., Wille, J., D'Armiento, J., and Chada, K. K. (1998). Epidermal expression of collagenase delays wound healing in transgenic mice. J. Invest. Dermatol. 111, 1029-1033; Duivenvoorden, W. C. M., Hirte, H. W., and Singh, G. (1997). Use of tetracycline as an inhibitor of matrix metalloproteinase activity secreted by human bone metastasizing cancer cells. Invasion and Metas. 17, 312-322; Fernandez-Catalan, C., Bode, W., Huber, R., Turk, D., Calvete, J. J., Lichte, A., Tschesche, H., and Maskos, K. (1998). Crystal structure of the complex formed by membrane type-I matrix metalloproteinase with the tissue inhibitor of metalloproteinases-2, the soluble progelatinase A receptor. EMBO J. 17, 5238-48; Freire, E., van Osdol, Mayorga, O L, and ánchez-Ruiz, J M. (1990). Calorimetrically determined dynamics of complex unfolding transitions in proteins. Annu Rev Biophys Biophys Chem. 19, 159-88; Gomis-Ruth, F. X., Maskos, K., Betz, M., Bergner, A., Huber, R., Suzuki, K., Yoshida, N., Nagase, H., Brew, K., Bourenkov, G. P., Bartunik, H., and Bode, W. (1997). Mechanism of inhibition of the human matrix metalloproteinase stromelysin-1 by TIMP-1. Nature 389, 77-81; Grams, F., Reinemer, P., Powers, J. C., Kleine, T., Pieper, M., Tschesche, H., Huber, R., Bode, W. (1995). X-ray structures of human neutrophil collagenase complexed with peptide hydroxamate and peptide thiol inhibitors: Implications for substrate binding and rational drug design. Eur. J. Biochem. 228, 830-834; Guex, N. and Peitsch, M. C. (1997). Swiss Model and the Swiss-PdbViewer: An environment for comparative protein modeling. Electrophoresis 18, 2714-2723; Higgins, D G., Bleasby, A J., and Fuchs, R. (1992). CLUSTAL V: improved software for multiple sequence alignment. Comput Appl Biosci., 8(2), 189-91; Howard, E. W., Bullen, E. C., and Banda, M. J. (1991). Preferential inhibition of 72 and 92 kDa gelatinase by tissue inhibitor of metalloproteinase-2. J. Biol. Chem. 266, 13070-13075; Huang, W., Suzuki, K., Nagase, H., Arumugam, S., Van Doren, S. R., and Brew, K. (1996). Folding and characterization of the amino terminal domain of human tissue inhibitor of metalloproteinases-1 (TIMP-1) expressed at high yield in E. coli. FEBS Lett. 384, 155-161; Karlsson, R., and Falt, A. (1997). Experimental design for kinetic analysis of protein-protein interactions with surface plasmon resonance biosensors. J. Immunol. Meths. 200, 121-33; Lakowicz, J. R. (1983). Principles of Fluorescence Spectroscopy, Chapter 10, Plenum Press, New York, London; Levit, S., and Berger, A. (1976). Ribonuclease S-peptide. A model for molecular recognition. J. Biol. Chem. 251, 1333-9; Levy, D. E., Lapierre, F., Liang, W., Ye, W., Lange, C. W., Li, X., Grobelny, D., Casabonne, M., Tyrrell, D., Holme, K., Nadzan, A., and Galardy, R. E. (1998). Matrix metalloproteinase inhibitors: A structure activity study. J. Med. Chem. 41, 199-223; Li, J., Brick, P., O'Hare, M. C., Skarzynski, T., Lloyd, L. F., Curry, V. A., Clark, I. M., Bigg, H. F., Hazleman, B. L., Cawston, T. E., et al. (1995). Structure of full-length porcine synovial collagenase reveals a C-terminal domain containing a calcium-linked, four-bladed beta-propeller. Structure 3, pp. 541-49; Libson, A. M., Gittis, A. G., Collier, I. E., Manner, B. L., Goldberg, G. I., and Lattman, E. E. (1995). Crystal structure of the haemopexin-like C terminal domain of gelatinase A. Nat. Struct. Biol. 2, 938-42; Lofas, S., Johnsson, B., Tegendahl, K., and Ronnberg, I. (1993). Dextran modified gold surfaces for surface plasmon resonance biosensors; immunoreactivity of immobilized antibodies and antibody-surface interaction studies. J. Colloid Interface Sci. 65, 423-431; Morton, T. A., Myska, D. G., and Chaiken, I. M. (1995). Interpreting complex binding kinetics from optical biosensors: A comparison of analysis by linearization, the integrated rate equation, and numerical integration. Anal. Biochem. 227, 176-185; Moses, M. A., Marikovsky, M., Harper, J. W., Vogt, P., Eriksson, E., Klagsbrun, M. and Langer, R. (1996). Temporal study of the activity of matrix metalloproteinases and their endogenous inhibitors during wound healing. J. Cell. Biochem. 60, 379-386; Odake, S., Morita, Y., and Morikawa, T. (1994). Inhibition of matrix metalloproteinases by peptidyl hydroxamic acids. Biochem. Biophys. Res. Comm. 199, 1442-1446; Olson, M. W., Gervasi, D. C., Mobashery, S., and Fridman, R. (1997). Kinetic analysis of the binding of human matrix metalloproteinase 2 and 9 to tissue inhibitor of metalloproteinase (TIMP)-1 and TIMP-2. J. Biol. Chem. 272, 29975-29983; O'Shannessy, D. J., Brigham-Burke, M., Soneson, K. K, Hensley, P., and Brooks, I. (1993). Determination of rate and equilibrium binding constants for macromolecular interactions using surface plasmon resonance: use of non linear least squares analysis methods. Anal. Biochem. 212, 457-468; Reinemer, P., Grams, F., Huber, R., Kleine, T., Schnierer, S., Pieper, M., Tschesche, H., Bode, W. (1994). Structural implications for the role of the N terminus in the superactivation of collagenases: A crystallographic study. FEBBS Lett. 338, 227-33; Saarialho-Kere, U. K. (1998). Patterns of matrix metalloproteinase and TIMP expression in chronic ulcers. Arch. Dermatol. Res. 290 (suppl), 47-54; Sayle, R. A. and Milner-White, E. J. (1995). RasMol: Biomolecular graphics for all. Trends in Biochemical Sciences 20, 374-376; Segel, I H. (1993) Enzyme Kinetics Behavior and analysis of rapid equilibrium and steady-state enzyme systems. Wiley Classics Library, John Wiley and Sons, Inc. New York; Su, J-L., Becherer, D., Edwards, C., Bukhart, W., McMgeehan, G. M., and Champion, B. R. (1995). Monoclonal antibodies against human collagenase and stromelysin. Hybridoma. 14, 383-390; Taylor, K. B., Windsor, J. L., Caterina, N. C. M., Bodden, M. K., and Engler, J. A. (1996). The mechanism of inhibition of collagenase by TIMP-1. J. Biol. Chem. 271, 23938-23945; Tuuttila, A., Morgunov, E., Bergmann, U., Lindqvist, Y., Maskos, K., Fernandez-Catalan, C., Bode, W., Tryggvason, K., and Schneider, G. (1998). Three dimensional structure of human tissue inhibitor of metalloproteinases-2 at 2.1.ANG. resolution. J. Mol. Biol. 284, 1133-1140; Vaalamo, M., Weckroth, M., Puolakkainen, P., Kere, J., Saarinen, P., Lauharanta, J., and Saarialho-Kere, U. K. (1996). Patterns of matrix metalloproteinase and TIMP-1 expression in chronic and normally healing human cutaneous wounds. Brit. J. Dermatol. 135, 52-59; Vaalamo, M., Mattila, L., Johansson, N., Kariniemi, A-L., Karjalainen-Lindsberg, -L., Kahari, V-M., and Saarialho-Kere, U. K. (1997). Distinct populations of stromal cells express collagenase-3 (MMP-13) and collagenase-1 (MMP-1) in chronic ulcers, but not in normally healing wounds. J. Investig. Dermatol. 109, 96-101; Weckroth, M., Vaheri, A., Lauharanta, J., Sorsa, T., and Konttinen, Y. T. (1996). Matrix metalloproteinases, gelatinases, and collagenases in chronic leg ulcers. J. Investig. Dermatol. 108, 1119-1124; Wojtowicz-Praga, S. M., Dickson, R. B., and Hawkins, M. J. (1997). Matrix metalloproteinase inhibitors. Investigational new Drugs. 15, 61-75.

These above references are included to show the great amount of prior art effort in this area, which has still not provided a satisfactory solution to this problem.

The present invention provides a method of prevention and/or treatment of ailments caused by or associated with dysfunction of MMP, which includes but not limited to inflammation or inflammatory responses, wound, acne, rosacea, skin aging, skin tone discoloration, skin wrinkles, dark skin, age spots, acne, loss of cellular antioxidants, loss of collagen, loss of skin pliability, loss of skin suppleness, oily skin, or a combination thereof.

Ubiquitin—Proteasome System.

The body constantly produces proteins and degrades proteins that are no longer needed or are defective. The production and destruction of proteins, called protein turnover, is a constant, ongoing process that is crucial for tissue renewal. A well-nourished person synthesizes nearly one pound of protein per day. Proteins that are broken down balance this protein gain. The process of protein breakdown, called proteolysis, is essential to cell survival. Numerous proteolytic systems exist in mammalian cells, the most important of which are the lysosomes, the ubiquitin—proteasome pathway, and enzymes called calpains. Lysosomes are small cell components that contain specific enzymes (proteases), which break down proteins. In the ubiquitin—proteasome pathway, proteins that are to be degraded are first marked by the addition of ubiquitin molecules and then broken down by large protein complexes called proteasomes. Calpains are proteases that are involved in several physiological processes, including the breakdown of proteins that give cells their shape and stability. The ubiquitin—proteasome system is now considered the major system involved in intracellular protein degradation. Two major components of this system are (1) three enzymes that add a small protein called ubiquitin onto substrate proteins destined for degradation, and (2) the proteasome, a rather large cellular particle composed of several smaller protein subunits, which executes the actual proteolysis. By degrading short-lived regulatory proteins, the ubiquitin—proteasome system controls basic cellular processes such as cell division, cell signaling, and gene regulation. The system also removes misfolded, damaged proteins, and in certain immune cells it breaks down foreign proteins into pieces called antigenic peptides, which can then be transported to the cell surface to induce an immune response [Ulrich, Current Topics in Microbiology and Immunology, vol 268, 137-174 (2002)].

Ubiquitin is a small protein that occurs in most eukaryotic cells. Its main function is to mark other intracellular proteins for destruction, known as proteolysis. Several ubiquitin molecules attach to the condemned protein (polyubiquitination), and polyubiquitinylated protein then moves to a proteasome, a barrel-shaped structure where the proteolysis occurs. Ubiquitin can also mark transmembrane proteins (for example, receptors) for removal from the membrane.

Ubiquitin consists of 76 amino acids with two sequentially linked glycine moieties at the carboxyl terminal and has a molecular mass of about 8500 amu (atomic mass units). It is highly conserved among eukaryotic species: Human and yeast ubiquitin share 96 percent amino acid sequence identity.

The process of marking a protein with ubiquitin consists of a series of steps; (1)

Activation of ubiquitin—the carboxyl group of the terminal glycine of ubiquitin binds to the sulfhydryl group —SH of an ubiquitin-activating enzyme E1. The sulfhydryl group is a cysteine residue on the E1 protein. This step requires an ATP molecule as an energy source and results in the formation of a thioester bond between ubiquitin and E1; (2) Transfer of ubiquitin from E1 to the ubiquitin-conjugating enzyme E2 via trans (thio) esterification; (3) Then, the final transfer of ubiquitin to the target protein can occur either directly from E2 (this is primarily used when ubiquitin is transferred to another ubiquitin already in place, creating a branched ubiquitin chain) or via an E3 enzyme, which binds specifically to both E2 and the target protein. The target protein is usually a damaged or non-functional protein that is recognized by a destruction-targeting sequence. Ubiquitins then bind to a lysine residue in the target protein via the transformation of thioester bond into an iso-peptide bond, eventually forming a tail of at least four ubiquitin molecules. The resulting ubiquitin-linked protein, called ubiquitin—protein conjugate, then can be recognized and degraded by the proteasome into peptides. This is the typical way to mark specific proteins for proteolysis. A functional proteasome (also called 26S proteasome) is composed of a smaller barrel-shaped core and two “caps” that are attached to the each end of the core. The proteasome core consists of four stacked rings containing two types of subunits, all facing into a central cavity. These subunits together have at least five distinct proteinase activities that cleave proteins at different sites. The “caps” at each end of proteasome perform a regulatory function. Each cap is composed of multiple subunits with numerous functions. These subunits recognize the ubiquitinylated protein, cut off the ubiquitin chains from this protein, thereby “unfolding” the protein, and open the channel inside the proteasome core so that th2 protein can enter the channel for degradation; and (4) Finally, the marked protein is digested in the 26S-proteasome into small peptides, amino acids (usually 6-7 amino acid subunits). Although the ubiquitins also enter the proteasome, they are not degraded (despite their protein structure) and may be used again.

Proteasomes are large multi-subunit protease complexes, localized in the nucleus and cytosol, which selectively degrade intracellular proteins. Proteasomes play a major role in the degradation of many proteins that are involved in cell cycling, proliferation, and apoptosis.

Intracellular proteolysis is the most recently discovered regulatory system of cellular physiology. Everything from cell division, development, and differentiation to cellular senescence has a proteolytic component. There is no simpler way to stop a physiological process than to destroy one of the components of a pathway in a controlled fashion. The discovery of the role of ubiquitin in the proteolytic pathway earned Aaron Ciechanover, Avram Hershko and Irwin Rose the 2004 Nobel Prize in Chemistry. Several books have become available that further reveal the importance of ubiquitins in human biology and human disease control, some of which are included herein for reference only: Ubiquitin and the Chemistry of Life, Mayer et al., John Wiley, 2005; Ubiquitin, Rechsteiner et al, Plenum Press, 1988; The Ubiquitin System, Schlesinger et al., Cold Spring Harbour Lab, 1988; Ubiquitins and the Biology of the Cell, Peters et al., Plenum Press, 2001; Self-Perpetuating Structural States in Biology, Disease, and Genetics (2002), Proceedings of the National Academy of Sciences.

A wide variety of neurodegenerative disorders are associated with the accumulation of ubiquitinylated proteins (if they are not further degraded by Proteasomes) in neuronal inclusions, and also with signs of inflammation. In these disorders, the ubiquitinylated protein aggregates, which will be seen as a foreign body by immune system, may themselves trigger the expression of inflammatory mediators, such as cyclooxygenase 2 (COX-2). Impairment of ubiquitin—proteasome pathway may contribute to this neurodegenerative and inflammatory processes. Products of COX-2, such as prostaglandin J2, can, in turn, increase the levels of ubiquitinylated proteins and also cause COX-2 up-regulation, creating a self-destructive feedback mechanism [Zongmin Li et al., International Journal of Biochemistry and Cell Biology, vol. 35, 547-552 (2003)].

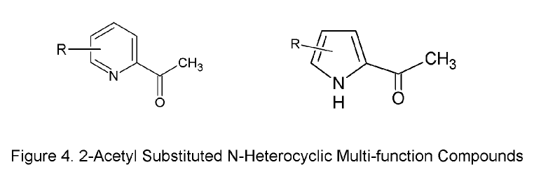

The disruption of the Ubiquitin—proteasome pathway can result from damaging events, such as aging-induced decrease in proteasome function [Carrard et al., International Journal of Biochemistry and Cell Biology, vol. 34, 1461 (2002)], oxidative stress [Shringarpure et al., Free Radical Biology Medicine, vol. 32, 1084-1089 (2002)], and production of neurotoxic molecules from mutations. A dysfunctional ubiquitin—proteasome pathway may then cause proteins that are normally turned over by this pathway to aggregate and form inclusions. One of the mechanisms by which the abnormal accumulation of ubiquitinylated proteins may mediate neurodegradation is by triggering an inflammatory response. Inflammation is a natural defense against diverse insults, intended to remove damaging agents and to inhibit their detrimental effects. Treatment of neurons with proteasome inhibitors, oxidative stressors, or cyclopentenone prostaglandin J2 elicits accumulation of ubiquitinylated proteins and cytotoxicity in a concentration-dependent manner. These agents also increased the neuronal levels of COX-2 and prostaglandin E2. COX-2 is the pro-inflammatory and inducible form of cyclooxygenases, which are enzymes that catalyze the rate-limiting step in the biosynthesis of prostaglandins, prostacyclins, or thromboxane A2 from their precursor arachidonic acid. Cyclooxygenases are bifunctional hemoproteins that catalyze the cyclooxygenation of arachidonic acid to PGG2 followed by the hydroperoxidation of PGG2 to PGH2. Specific enzymes, such as reductases, isomerases, and synthases, then convert PGH2 to other PGs (prostaglandins) and thromboxane A2. Reactive oxygen species (ROS) produced during this biosynthetic pathway are known to contribute to tissue damage. The pro-oxidant effect of prostaglandin J2 could me mediated by its cyclopentenone ring that contains an alpha-beta-unsaturated carbonyl group that can react with sulfhydryl group of cysteine residues in glutathione and cellular proteins to inhibit ubiquitin isopeptidase activity. This may also contribute to the accumulation of ubiquitinylated proteins. This toxic positive feedback may create a self-destructive mechanism that contributes to the neurodegenerative process (FIG. 4). Neurodegenerative disorders, such as Alzheimer's disease, Parkinson's disease, and amylotropic lateral sclerosis, found to be associated with the accumulation of ubiquitinylated proteins in neuronal inclusions also exhibit signs of inflammation. Ross et al. [Trends Cell Biol., vol. 14(12):703-11 (2004)] provide a detailed discussion of the ubiquitin—proteasome pathway in Parkinson's disease and other neurodegenerative diseases. Burger et al. [Eur. J. Cancer., vol. 40(15):2217-29 (2004)] provide an insight into the ubiquitin-mediated protein degradation pathway in cancer therapeutic implications. A book edited by Peters et al, “Ubiquitin and the Biology of the Cell”, Plenum Publishing, provides information on the importance of ubiquitin in modulating cellular functions.

The modulation of ubiquitin—proteasome pathway can be achieved in several manners that includes, (1) the inhibition of thioester bond formation between ubiquitin and cysteine moiety of ubiquitin activating enzyme (E1, E2, or E3), (2) the inhibition of iso-peptide bond formation between ubiquitin and lysine moiety of target protein (3) the inhibition of ubiquitin—proteasome complex, (4) acceleration of proteolysis by ubiquitin—proteasome complex (acceleration of proteasome ligase, E3, action), (5) selective inhibition of cyclooxygenase enzyme, (6) use of thiol reducing antioxidants, (7) caspase inhibitors, and (8) use of molecular chaperones to attenuate the accumulation of ubiquitinylated proteins. The molecular chaperones could thus be highly beneficial in the reduction of inflammation caused by accumulating ubiquitin—proteasome complex, which could be useful for the treatment of skin aging, inflammation, ulcer and wound healing, and enzyme malfunction related ailments.

The present invention relates to a method of modulation of ubiquitin—proteasome pathway enzymes.

Dysfunction of Epidermal melanocyte-regulating Enzymes.

The color of human skin is differentiated by the nature and quantity of natural pigment, melanin, present in the epidermal layers of skin. The formation of melanin from amino acid tyrosine involves several biogenetic steps mediated initially by enzyme tyrosinase. Tyrosinase is a copper-based monooxygenase enzyme that catalyzes the hydroxylation of monophenols (hydroxybenzenes) and the oxidation of ortho-diphenols to ortho-quinones. This enzyme, found in prokaryotes as well as in eukaryotes, is involved in the formation of pigments such as melanins and other polyphenolic compounds. The active-site of tyrosinase is known to contain two copper ions (CuA and CuB). Each of the two copper ions is bound by three conserved histidine residues. The regions around these copper-binding ligands are well conserved. Moreover, the distance between these two copper ions is 26 Angstrom units [(van Amsterdam et al., Angewandte Chemie, 42: 62-64 (2003); Bubacco et al., J. Biol. Chem., 181-194 (2003)]. At least two proteins related to tyrosinase are known to exist in mammals, and include TRP-1, which is responsible for the conversion of 5,6-dihydroxyindole-2-carboxylic acid (DHICA) to indole-5,6-quinone-2-car-boxylic acid (IQCA) or indole-5,6-quinone (IQ); and TRP-2, which is the melanogenic enzyme DOPAchrome tautomerase that catalyzes the conversion of DOPAchrome to DHICA. TRP-2 differs from tyrosinases and TRP-1 in that it binds two zinc ions instead of copper. Other proteins that belong to this family are plant polyphenol oxidases (PPO), which catalyze the oxidation of mono- and ortho-diphenols to ortho-diquinone. From this discussion it should be clearly evident that any successful inhibition of tyrosinase at its active-site level is to be accomplished only by the blocking of both copper and zinc actives-sites. This should be possible by the use of appropriate transition state analogs. Moreover, any changes in the environment of copper-copper linkage at the active-site of tyrosinase that can result in the distortion of 26 Angstrom distance between those two copper atoms can also cause a disruption in the enzymatic activity of tyrosinase.

The first step of tyrosinase action is the most critical because the remainder of the reaction sequence can proceed spontaneously at physiological pH. Here, tyrosinase converts tyrosine to dihydroxyphenylalanine (DOPA) and then to dopaquinone. Subsequently, dopaquinone is converted to dopachrome, through auto-oxidation, and finally to dihydroxyindole (DHI) or dihydroxyindole-2-carboxylic acid (DHICA), which form eumelanin (brown-black pigment). The latter reaction occurs in the presence of dopachrome tautomerase and DHICA oxidase. In the presence of cysteine or glutathione, dopaquinone is converted to cysteinyl DOPA or glutathione DOPA. Subsequently, pheomelanin, a yellow-red pigment, is formed. This is further discussed in U.S. Pat. No. 5,679,511 (Kwon). The level of melanin present in the epidermis and hair fiber determines skin and hair pigmentation. For example, three different types of melanin are present in the epidermis: DHI-melanin, DHICA-melanin and pheomelanin. The different types of melanin vary in color or shade. DHI-melanin is the darkest and is blackish in color. DHICA-melanin is brownish in color. Pheomelanin is the lightest and is reddish in color. Pheomelanin is produced by the entrapment of dopachrome by sulfur-containing species such as cysteine and glutathione.

The inhibition of melanin biosynthesis may be achieved by the following mechanisms: (1) The competitive replacement of tyrosinase substrates, tyrosine or 1-dopa with other chemically related compositions, (2) The inhibition of the oxidation/hydroxylation of tyrosine to produce 1-dopa, (3) The inhibition of the conversion of 1-dopa into dopaquinone, (4) The inhibition of the conversion of dopaquinone into dopachrome, (5) The inhibition of the conversion of dopachrome into DHICA or DHI, (6) The inhibition of the conversion of DHICA or DHI into IQCA or IQ, (7) The irreversible inactivation, replacement, or change in the metal-to-metal oxidation state of copper and zinc ion active-sites of TRP-1 and TRP-2. These mechanisms lead to the blocking of dark colored Eumelanin. An additional process to reduce the formation of darker colored Eumelanin is to promote the formation of Pheomelanin. For example, in the presence of cysteine or glutathlone, dopaquinone can be converted to cysteinyl DOPA or glutathione DOPA, which lead to the formation of less dark colored pheomelanin, (8) Changes in the environment of copper-copper linkage at the active-site of tyrosinase that can result in the distortion of 26 Angstrom distance between those two copper atoms, and (9) inhibition of melanocyte stimulating hormone (MSH). It has been generally known to utilize a tyrosinase inhibitor or a tyrosine competitor (to block enzyme tyrosinase), or a reducing agent (to convert melanin and its pigmented precursors into colorless, or less colored biochemical entities) by the prior art skin whitening compositions. A comprehensive treatment that encompasses a combination of the above biochemical mechanisms has been unknown.

A great number of skin whitening compositions have become commercially available. Most of those preparations are tyrosinase inhibitors. For example, Gattefosse markets “Gatulin Whitening”, which is a mixture of Aspergillus Orizae and Licorice extracts. Gattefosse also markets “Synerlight”, which is a mixture of Sophora root extract, kiwi water, and ascorbic acid, and “Mulberry Extract”, for skin whitening applications. “Etioline”, which is a mixture of Mitracarpus scaber extract and bearberry (Arctostaphylus uva ursi) extract, is skin whitening tyrosinase inhibitor marketed by Sederma. “Melaslow” and “Melaclear” are two additional skin-whitening compositions marketed by Sederma, both of which are based on tyrosinase inhibitors. “Dermalight” is a tyrosinase inhibiting composition based on nasturtium petals marketed by Silab. “Clariskin”, also offered by Silab, is another tyrosinase inhibitor derived from wheat germ extract. “Tyrostat-09” and “Tyrostat-11” marketed by Dragoco are both based on tyrosinase inhibitors obtained from a Canadian plant, and further disclosed in U.S. Pat. No. 6,521,267 (Steck). Alpaflor offers “Glgawhite”, a skin whitening composition based on a mixture of several tyrosinase inhibiting botanical extracts including Malva sylvestris, Mentha piperita, Primula veris, Alchemilla vulgaris, Veronica officinalis, Melissa offlcinalis, and Achillea millefolium. A much smaller number of compositions are available that generally act by reducing mechanism. The examples include hydroquinone, arbutin, and ascorbic acid and its derivatives.

Hydroquinone (HQ). An important industrial chemical, HQ is also a ubiquitous chemical readily available in cosmetic and nonprescription forms for skin lightening. it is considered one of the most effective inhibitors of melanogenesis in vitro and in vivo. HQ causes reversible inhibition of cellular metabolism by affecting both DNA and RNA synthesis. The cytotoxic effects of HQ are not limited to melanocytes, although the dose required to inhibit cellular metabolism is much higher for nonmelanotic cells than for melanocytes. Thus, HQ can be considered a potent melanocyte cytotoxic agent with relatively high melanocyte-specific cytotoxicity. HQ is also a poor substrate of tyrosinase, thereby competing for tyrosine oxidation in active melanocytes. The 2 percent HQ is readily available over-the-counter in various cosmetic preparations. However, for better efficacy, it often is compounded into various mixtures for treatment of hyperpigmentation. The original Kligman formula involves compounding 5 percent HQ with 0.1 percent retinoic acid and 0.1 percent dexamethasone in a hydrophilic ointment base. Concentrations as high as 1 Opercent can be compounded extemporaneously for refractory cases. Evidence of improvement with HQ (monotherapy) usually is observed at 4-6 weeks, with improvement appearing to plateau at about 4 months.

Monobenzyl ether of Hydroquinone. Like HQ, monobenzyl ether of hydroquinone (MBEH) belongs to the phenol/catechol class of chemical agents. However, unlike HQ, MBEH almost always causes nearly irreversible depigmentation of skin. Traces of MBEH have been found in disinfectants, germicides, rubber-covered dish trays, adhesive tape, powdered rubber condoms, and rubber aprons. In dermatology, MBEH should be used only to eliminate residual areas of normally pigmented skin in patients with refractory and generalized vitiligo. It has been suggested that the mechanism of depigmentation of MBEH is because of the selective melanocytic destruction through free radical formation and competitive inhibition of tyrosinase enzyme system.

U.S. Pat. No. 4,526,179 refers to certain hydroquinone fatty esters that have good activity and are less irritating and more stable than hydroquinone. Japanese Patent Application No. 27909/86 refers to other hydroquinone derivatives that do not have the drawbacks of hydroquinone but that have relatively poor efficacy. U.S. Pat. No. 5,449,518 refers to 2,5-dihydroxyphenyl carboxylic acid derivatives as skin depigmentation agents. However, it should be noted that several hydroquinone derivatives are potent allergens. For example primrose (Primula obconica) contains hydroquinone derivative, Miconidin (2-methoxy-6-pentyl-1,4-dihydroxybenzene) and its oxidation product, Primin (2-methoxy-6-pentyl-1,4-benzoquinone), both of which are allergens [(Peng Nan et al., Annals of Botany, 91: 329-333 (2003)]. From the same plant methyl 2,4-dihydroxy-5-methyl benzoate (30.41 percent), methyl 2,6-dihydroxy-4-methyl benzoate (29.27 percent), and hypnone (8.92 percent) were also obtained, all of which were non-allergenic [(Na P et al., Nat Prod Letters, 16(4):249-53 (2002)]. in another species of primrose (Primula ovalifolia), Peng Nan et al. [(Z. Naturforsch. 58, 57-61 (2003)] reported the occurrence of acetyl hydroquinone and methyl acetyl hydroquinone, both of which were not studied for possible skin whitening effects by these authors.

N-Acetyl-4-S-cysteaminylphenol: Like HQ and MBEH, N-acetyl-4-S-cysteaminylphenol (4-S-CAP) belongs to the class of phenol/catechols. The acetyl derivative of 4-S-CAP appears to be an excellent substrate of tyrosinase substrate; it forms a melanin-like pigment when exposed to tyrosinase. Like HQ, it also is considered to be cytotoxic. in a study of 12 patients with melasma who used 4 percent 4-S-CAP, Jimbow [(Arch Dermatology, 127(10):1528-34 (1991)] reported a 66 percent improvement after 4 weeks of use. Furthermore, the author reported it to be more stable and less irritating than HQ.

Azelalc acid: A naturally occurring, saturated dicarboxylic acid originally isolated from Pityrosporum ovale, azelaic acid is a rather weak competitive inhibitor of tyrosinase in vitro. In addition, azelaic acid has an antiproliferative and cytotoxic effect on melanocytes. The latter effect is because of a rather potent inhibition of thioredoxin reductase, an enzyme involved in mitochondrial oxidoreductase activation and DNA synthesis. Azelaic acid is prescribed topically as a 20 percent cream and has been combined with glycolic acid (15 percent and 20 percent), and its efficacy has been compared with HQ 4 percent in the treatment of facial hyperpigmentation in dark-skinned patients. it has been reported that the combination formula was as effective as HQ 4 percent cream, although with a slightly higher rate of local irritation.

Kojic acid (5-hydroxy-2-methyl-4-pyran-4-one): A fungal metabolic product, kojic acid inhibits the catecholase activity of tyrosinase, which is the rate-limiting, essential enzyme in the biosynthesis of the skin pigment melanin. Kojic acid also is consumed widely in the Japanese diet with the belief that it is of benefit to health. Indeed, it has been shown to significantly enhance neutrophil phagocytosis and lymphocyte proliferation stimulated by phytohemagglutinin. Melanocytes treated with kojic acid become nondendritic with decreased melanin content. Additionally, it scavenges reactive oxygen species that are released excessively from cells or generated in tissue or blood. Kojic acid is used in concentrations ranging from 1-4 percent. Although effective as a skin-lightening gel, it has been reported to have high-sensitizing potential and cause irritant contact dermatitis. in a study comparing glycolic acid/kojic acid combination with glycolic acid/HQ, no statistical difference in efficacy existed between kojic acid and HQ. However, the kojic acid preparation was reported to be more irritating.

4-Hydroxyanisole: Like HQ, 4-hydroxyanisole (4HA) is cytotoxic to melanocytes. its clinical efficacy in inhibiting melanogenesis has been reported when used as a combination of 4HA 2 percent cream and 0.01 percent retinolc acid. The authors reported minimal local skin irritation with this combination. 4HA 2 percent alone did not produce significant hypo pigmentation.

Arbutin (hydroquinone-beta-D-glucopyranoside): A glycosylated HQ found at high concentrations in certain plants that are capable of surviving extreme and sustained dehydration, arbutin has been shown to inhibit melanin synthesis by inhibition of tyrosinase activity. This appears to be because of the inhibition of melanosomal tyrosinase activity, rather than the suppression of the synthesis and expression of this enzyme. Because arbutin does not hydrolyze to liberate HQ, the latter agent is not responsible for the inhibitory effect of arbutin on melanogenesis. Although the effective topical concentration in treating disorders of hyperpigmentation has not been formally evaluated and published, several manufacturers are marketing arbutin as a depigmenting agent.

Paper Mulberry: This tyrosinase inhibitor was isolated from a plant herbal extract. The plant roots from which paper mulberry was isolated were collected in Korea. The authors compared the tyrosinase inhibition of paper mulberry to kojic acid and HQ, the concentration causing 50 percent inhibition of the activity of tyrosinase, was reported to be 0.396 percent compared to 5.5 percent for HQ and 10.0 percent for kojic acid. The authors also performed a patch test using 1 percent paper mulberry extract and found no significant irritation at either 24 hours or 28 hours.

Glabridin: Glabridin is the main ingredient in licorice extract. The authors investigated glabridin for its inhibitory effect on pigmentation and reported that glabridin inhibited tyrosinase activity of melanocytes without any cytotoxicity. They further showed that UV-Binduced pigmentation and erythema was inhibited by topical application of 0.5 percent glabridin. The anti-inflammatory properties of glabridin were attributed to inhibition of superoxide anion production and cyclooxygenase activity.

Arctostaphylos patula and Arctostaphylos viscida: The leaves of these two Arctostaphylos plants have been reported to be potent inhibitors of tyrosinase. These two extracts not only inhibited the production of melanin from dopachrome but also exhibited superoxide dismutase-like activity. The effective topical concentration of these two plants in disorders of hyperpigmentation currently is not known.

Melatonin: Melatonin is secreted by the pineal gland in response to sunlight. This pineal agent is considered to be responsible for lightening the color of amphibians. When added to cultures of hair follicles of the Siberian hamster, melatonin was shown to bring about a dose-related inhibition of melanogenesis. However, tyrosinase activity was not affected, suggesting that the inhibition of melanogenesis occurs at the post-tyrosinase step in the melanin biosynthetic pathway. Melatonin has been shown to inhibit adenosine 3′,5′-cyclic phosphate (cAMP) driven processes in pigment cells. The concentration for topical use of melatonin for hyperpigmentation disorders has not been formally established. However, topical melatonin also has been reported to have anti-inflammatory properties when applied at 0.6 mg/cm. A cosmetic manufacturer currently producing and marketing topical melatonin cream reports melatonin as an effective antioxidant when topically applied at a concentration of 1 percent.

Magnesium ascorbyl phosphate: Magnesium ascorbyl-2-phosphate (MAP) is a stable derivative of ascorbic acid. When used as a 1 Opercent cream, MAP was shown to suppress melanin formation. A significant lightening effect was seen clinically in 19 of 34 patients with melasma and solar lentigos. Furthermore, MAP has been shown to have a protective effect against skin damage induced by UV-B irradiation. The latter protective effect is because of the conversion of MAP to AS.

Surprisingly and unexpectedly, a method of treatment has now been found that comprises topical application of certain hydroxyaryl compounds that contain an alkyl carbon side chain with a ketone group attached at the first carbon atom of the alkyl side chain, and said ketone group is directly attached to the aromatic ring at a position adjacent to hydroxyl group of hydroxyaryl ring; which now provides a multi-function treatment of dysfunction of three key enzymes: (i) extra-cellular matrix metalloproteases, and (ii) intra-cellular ubiquitin—proteasomes, and (iii) epidermal melanocyte-regulating enzymes. Additionally, the method of the present invention provides treatment of skin disorder caused by dysfunction of said dermal enzymes; wherein said skin disorder is skin aging, skin wrinkles, dark skin, age spots, acne, skin inflammation, loss of cellular antioxidants, loss of collagen, loss of skin pliability, loss of skin suppleness, oily skin, or a combination thereof.

A number of said hydroxyaryl compounds obtained from natural plant sources have been disclosed in the prior art with antioxidant and other benefits. For example, acetophenone derivatives such as Paeonol (3-hydroxy-5-methoxy acetophenone), 2,5-Dihydroxy-4-Methoxy Acetophenone, and 2,5-Dihydroxy-4-Methyl Acetophenone, have been obtained from Chinese peony. Quinacetophenone (2-acetyl hydroquinone) has been obtained from primrose (Primula Ovalifolia). Scutellarin and Scutellarein (hydroxy benzopyranones) have been obtained from Scutellaria plants. Xanthoxyline (2-hydroxy-4,6-dimethoxyacetophenone) has been isolated from Sebastiania schottiana. Acetophenone derivatives, such as 1-(3-Hydroxy-4-methoxy-5-methylphenyl)ethanone and 1-(3-hydroxy-4-methoxyphenyl)ethanone have been identified from stem bark of Lamprothamnus zanguebaricus. Apocynin (4-hydroxy-3-methoxyacetophenone), is a well-known acetophenone derivative isolated from the traditional medicinal plant Picrorhiza kurroa. 4-Hydroxyacetophenone has been obtained from Ligularia vellerea. These hydroxyaryl compounds are known in the prior art for their antioxidant, microcirculation improvement, anti-inflammatory, Mono Amine Oxidase (MAO) inhibition, and histamine suppression benefits. None of them have been reported for their multi-enzyme regulating or treating methods in the prior art.

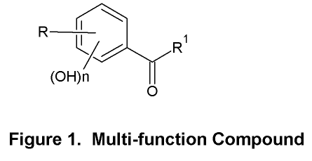

The topical method of treatment of the present invention comprises;
(i) An Extra-cellular, Matrix metalloprotease regulating agent, and
(ii) An Intra-cellular Ubiquitin—Proteasome regulating agent, and
(iii) An epidermal melanocyte-regulating agent, and, wherein,
(iv) Said extra-cellular agent, said intracellular agent, and said epidermal agent is a multi-function compound in accordance to [FIG. 1]:

[FIG. 1],
Wherein;
(OH)ₙ is one, two, or three OH substituents, one of which is 2-hydroxy;
R is one, two, or three substituents each independently selected from the group consisting of H, Alkyl, Cycloalkyl, Aralkyl, Aryl, Cl, Br, NH₂, NH-Alkyl, N(Alkyl)₂, O-Alkyl, S-Alkyl, Heterocyclic, and Heteroaryl; and
R¹ is selected from the group consisting of Methyl, Ethyl, Alkyl, Aralkyl, Heterocyclic, and Heteroaryl; and, wherein,
(v) Said complex is applied topically at a desired site in a sufficient quantity; and, wherein
(vi) Said application having been done either by a manual or a mechanical method, or a combination thereof; and, wherein
(vii) Said topical application causes the desired treatment of said skin condition or disorder.

The hydroxyaryl compound can be further selected from 2-hydroxyacetophenone, 3-hydroxyacetophenone, 4-hydroxyacetophenone, 2,3-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, 2-Acetyl resorcinol, 4-Acetyl resorcinol, 3,4-Dihydroxyacetophenone, Quinacetophenone, 1-(3-Hydroxy-4-methoxy-5-methylphenyl)ethanone, 1-(3-hydroxy-4-methoxyphenyl)ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, 2-hydroxypropiophenone, 3-hydroxypropiophenone, 4-hydroxypropiophenone, 2,3-dihydroxypropiophenone, 2,4-dihydroxypropiophenone, 2,5-dihydroxypropiophenone, 2,6-dihydroxypropiophenone, 3,4-dihydroxypropiophenone, 3,5-dihydroxypropiophenone, 2,4,6-trihydroxypropiophenone, 2,3,4-trihydroxypropiophenone, 2,3,5-trihydroxypropiophenone, 2,3,6-trihydroxypropiophenone, 2,4,5-Page 26 of 26 trihydroxypropiophenone, 3,4,5-trihydroxypropiophenone, phloridzin, phloretin, 1-(2,4-dihydroxyphenyl)-2-hydroxyethanone, (2-hydroxyphenyl)(oxo)acetic acid, 1-(2,6-dihydroxyphenyl)-1-butanone, 1-(1-hydroxy-2-naphthyl)ethanone, 1-(2-hydroxy-1-naphthyl)ethanone, 5,7-dihydroxy-1-indanone, 1-(2-hydroxy-5-methylphenyl)-1,3-butanedione, N-(4-acetyl-3-hydroxyphenyl)acetamide, 4-acetyl-3-hydroxyphenyl acetate, 1,1′-(4,6-Dihydroxy-1,3-phenylene)bisethanone, 1-(1-hydroxy-2-naphthyl)ethanone, 2,3-Dihydro-9,10-dihydroxy-1,4-anthracenedione, and combinations thereof.

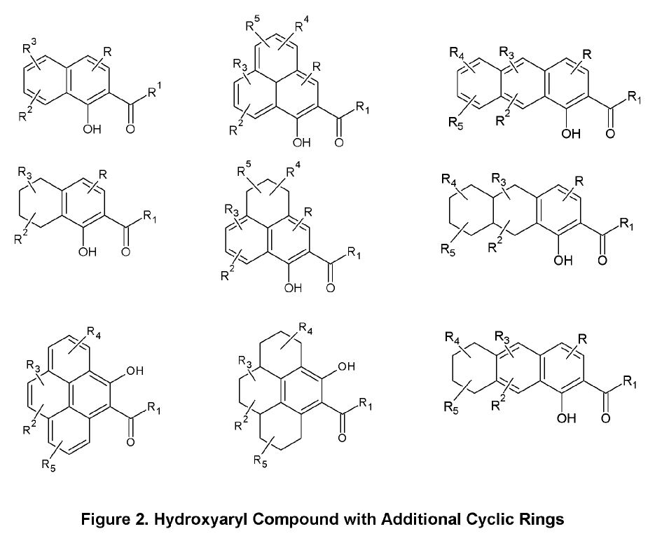

In the method of treatment of the present invention said multi-function compound can have additional cyclic rings attached at the aromatic moiety. Such attached rings can be alicyclic, aromatic, heteroaryl, heterocyclic, or a combination thereof, examples of which include 1-hydroxy-2-acetylnaphthalene; 1-hydroxy-2-acetyl-5,6,7,8-tetrahydro-naphthalene; 7-acetyl-8-hydroxyquinoline; 3-acetyl-4-hydroxyacridine; 6-acetyl-7-hydroxybenzothiazole. As can be appreciated by any one versed in the art that a very large number of compounds that have the structural criteria discovered in the present invention is possible [FIG. 2].

[FIG. 2]

Wherein;
R¹ is Methyl or Ethyl;
R², R³, R⁴, and R⁵ are each independently selected from the group consisting of H, OH, Methyl, Alkyl, Cyclo-Alkyl, Aryl, Cl, Br, NH₂, NH-Alkyl, N(Alkyl)₂, O-Alkyl, and S-Alkyl.

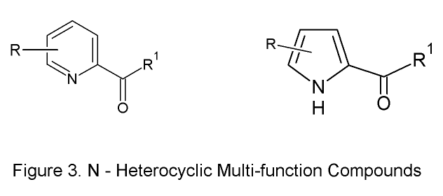

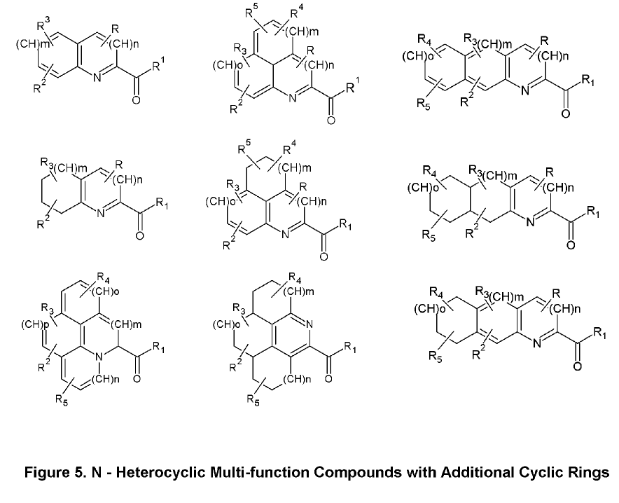

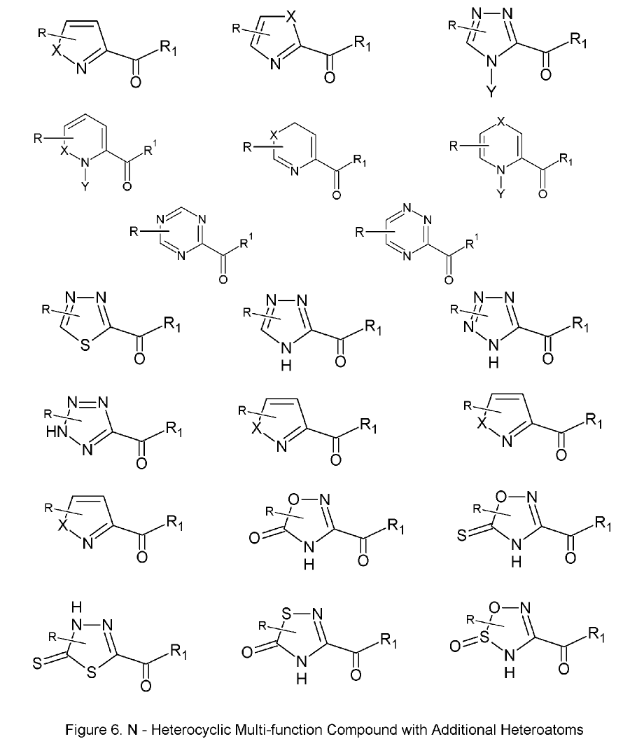

The multi-function compound can also have an attached nitrogen hereto-aromatic ring at a position adjacent to the ring nitrogen atom. Such compounds also show selective MMP inhibitory effect; as such compounds can also bind with zinc cation of the active site and cause distortion of the spatial configuration of the active site. Such spatial distortions cause an inhibitory effect for MMP activity. The five- and six-member hetero-aromatic ring of the acyl- or alkyl ketone-substituted MMP inhibitors of the present invention can have additional heteroatoms in their ring structure. For example, additional nitrogen atoms, or sulfur or oxygen atoms, or a combination thereof, can additionally be present. The examples of hetero-aromatic ring structures include 2-acetylpyridine, 2-acetylpyrrole, 2-acetylimidazole, 2-acetylthiazole, 2-acetylpyrimidine, 2-acetylindole, 2-acetyl-1-methylpyrrole, 2-acetyl-4-methylpyridine, 1-acetylphenothiazine, 2-hydroxy-1-acetylphenothiazine, 8-hydroxy-9-acetylphenanthrene, 2-acetylpyrazine, 2-acetylquinoline, 2-acetyl-8-hydroxyquinoline, 2-acetyltryptophane, 2-acetyltryptophanamide, 2-acetylpyridine N-oxide, 2-acetylquinazoline, 2-acetylquinoxaline, 3-acetylpyridazine, 6,6′-diacetyl-2,2′-pyridyl, 3-actyl-1,2,4-trizol, and their other acetyl side chain substituted and/or hetero-aromatic ring substituted derivatives. A specific example of this is 2-acetyl-8-hydroxyquinoline. A large variation in five- and six-member multi-heteroatom ring structures is thus possible, a select number of which are illustrated in [FIGS. 3, 4, 5, and 6]:

[FIG. 3]
Wherein:
R is one, two, or three substituents each independently selected from the group consisting of H, Alkyl, Cycloalkyl, Aryl, Cl, Br, NH₂, NH-Alkyl, N(Alkyl)₂, OH, O-Alkyl, and S-Alkyl; and
R¹ is Methyl, Ethyl, Alkyl, and Aryl.

[FIG. 4]
Wherein:
R is one, two, or three substituents each independently selected from the group consisting of Alkyl, Cycloalkyl, Aryl, Cl, Br, NH₂, NH-Alkyl, N(Alkyl)₂, OH, O-Alkyl, and S-Alkyl.

[FIG. 5]
Wherein:
R, R¹, R², R³, R⁴, R⁵ is one, two, or three substituents each independently selected from the group consisting of Alkyl, Cycloalkyl, Aryl, Cl, Br, NH₂, NH-Alkyl, N(Alkyl)₂, OH, O-Alkyl, and S-Alkyl; and
R¹ is Methyl, Ethyl, Alkyl, and Aryl;
n=0, or 1;
m=0, or 1;
o=0, or 1; and
p=0, or 1.

[FIG. 6]
Wherein;
R is one, two, or three substituents each independently selected from the group consisting of H, Alkyl, Cycloalkyl, Aryl, Cl, Br, NH₂, NH-Alkyl, N(Alkyl)₂, OH, O-Alkyl, and S-Alkyl;
R¹ is selected from the group consisting of Methyl, Ethyl, Alkyl, and Aryl;
X is selected from the group consisting of N, O, and S; and
Y is selected from the group consisting of H, Alkyl, Cycloalkyl, and Aryl.

In another preferred aspect, the method of the present invention can include a delivery system or a carrier base, which can be selected from, among others, a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and similar delivery or packaging form.

In another preferred aspect, the delivery system can be selected from, among others, traditional water and oil emulsions, suspensions, colloids, microemulsions, clear solutions, suspensions of nanoparticles, emulsions of nanoparticles, or anhydrous compositions.

Additional cosmetically or pharmaceutically beneficial ingredients can also be included in the method of the present invention, which can be selected from, but not limited to skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients or compositions can be selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

Certain divalent and polyvalent metal ions can also be present in the compositions of the present invention. The examples of such metal ions include zinc, copper, manganese, vanadium, chromium, cobalt, and iron.

### EXAMPLES

The following examples are presented to illustrate presently preferred practice thereof. These examples also include the formulation of consumer desirable lotion, cream, and other such compositions for their topical application in accordance to the method of the present invention. As illustrations they are not intended to limit the scope of the invention. All quantities are in weight percent.

### Example 1

Enzyme Dysfunction Serum. Ingredients percent Weight (1) Deionized water 20.0 (2) 2-Acetyl-8-hydroxyquinoxaline 5.0 (3) Methylpropanediol 69.5 (4) Dimethicone copolyol 4.0 (5) Preservatives 0.5 (6) Ammonium Acryloyldimethyltaurate/VP copolymer 1.0. Procedure. Make main batch by mixing (2) to (5) at room temperature. Pre-mix (1) and (6) to a clear paste and add to main batch with mixing. The product has a clear to slightly hazy syrup-like appearance, typical of a skin serum product. Upon topical application according to the method of the present invention is absorbed rapidly with a silky smooth skin feel.

### Example 2

Wound Healing Serum with Copper Ions. Ingredients percent Weight (1)

Deionized water 20.0 (2) Quinacetophenone 5.0 (3) Methylpropanediol 69.0 (4) Dimethicone copolyol 4.0 (5) Preservatives 0.5 (6) Copper Gluconate 0.5. (7) Ammonium Acryloyldimethyltaurate/VP copolymer 1.0 Procedure. Make main batch by mixing (2) to (6) at room temperature. Pre-mix (1) and (7) to a clear paste and add to main batch with mixing. The product has a clear to slightly hazy syrup-like light blue appearance, typical of a skin serum product. Upon topical application to the wound area according to the method of the present invention it is absorbed rapidly.

### Example 3

Wound Healing Cream. Ingredients percent Weight (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Resacetophenone 3.0 (7) Paeonol 1.0 (8) (8) Preservatives 0.5. Procedure. Mix 1 to 5 and heat to 75-80° C. Adjust pH to 4.0 4.5. Cool to 35-40 C with mixing. Add 6 to 8 with mixing. Adjust pH to 4.0-4.5, if necessary. White to off-white cream.

### Example 4

Collagen Boosting Antiaging Facial Mask Composition. Ingredient. (1) Chitosan 5.0 (2) 2,5-Dihydroxy acetophenone Oxime 5.0 (3) Glycerin 17.7 (4) Water 70.6 (5) Yohimbine HCl 0.5 (6) Niacinamide Lipoate 0.5 (7) Glutathione0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck according to the method of the present invention and left for 10 to 30 minutes, and then rinsed off.

### Example 5

Skin Discoloration and Age Spots Cure Cream. Ingredient percent (1) Water 65.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Niacinamide Lactate 1.0 (16) Niacinamide Hydroxycitrate 3.1 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Polysorbate-20 2.0 (23) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained. It is applied topically according to the method of the present invention.

### Example 6

Anti-inflammatory Acne Cream. Ingredient percent (1) Water 62.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Niacinamide Salicylate 4.0 (16) Niacinamide Hydroxycitrate 2.2 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Pyridoxine Salicylate (23) Polysorbate-20 2.0 (24) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained. It is applied topically according to the method of the present invention.

### Example 7

Anti-inflammatory Skin Brightening Cleanser. Ingredient percent (1) PEG-6 63.329 (2) Hydroxypropyl Cellulose 0.3 (3) Boswellia Serrata 0.05 (4) Sodium Cocoyl Isethionate 20.0 (5) Sodium Lauryl Sulfoacetate 5.0 (6) L-Glutathione 0.01 (7) Resveratrol 0.01 (8) 2,5-Dihydroxy Acetophenone 0.1 (9) 2,6-Dihydroxy Acetophenone 0.001 (10) Ascorbic acid 10.0 (11) Phenoxyethanol 0.7 (12) Ethylhexylglycerin 0.3 (13) Fragrance 0.2. Procedure. Mix (1) and (2) to a clear thin gel. Add all other ingredients and mix in a homogenizer. A white cream-like cleanser is obtained. It is applied topically according to the method of the present invention.

### Example 8

Arthritis Pain Relief Anti-inflammatory Gel. Ingredients percent (1) C12-15 Alkyl Benzoate 67.75 (2) Ethylenediamine/Hydrogenated Dimer Dilinoleate Copolymer Bis-Di-C14-18 Alkyl Amide 10.0 (3) Ximenia Oil 0.1 (4) Capsaicin 0.25 (5) Magnolol (and Honokiol 0.2 (6) Paeonol 0.5 (7) Tetrahydrocurcuminoids 0.2 (8) Zeolite 20.0 (9) Fragrance 1.0. Procedure. Mix (1) and (2) and heat at 80 to 90 C till clear. Cool to 40 to 50 C and add all other ingredients and mix. Cool to room temperature. A white gel-like product is obtained. It is applied topically according to the method of the present invention.

### Example 9

Arthritis Anti-inflammatory Transparent Gel. Ingredients percent (1) C12-15 Alkyl Benzoate 96.75 (2) Dibutyl Lauroyl Glutamide 1.0 (3) Ximenia Oil 0.1 (4) Capsaicin 0.25 (5) Magnolol (and Honokiol 0.2 (6) Paeonol 0.5 (7) Tetrahydrocurcuminoids 0.2 (8) Fragrance 1.0. Procedure. Mix (1) and (2) and heat at 95 to 110 C till clear. Cool to 40 to 50 C and add all other ingredients and mix. Cool to room temperature. A transparent gel-like product is obtained. It is applied topically according to the method of the present invention.

### Example 10

Topical Anesthetic Spray Lotion with Anti-inflammatory Agents. Ingredients percent (1) PEG-4 81.0 (2) Benzocaine 16.0 (3) Fragrance 0.5 (4) Paeonol 0.5 (5) 2,4-Dihydroxy Acetophenone 2.0. Procedure. Mix all ingredients in a clear solution is obtained. Fill in spray bottles. It is applied topically according to the method of the present invention.

### Example 11

Anti-inflammatory Color-Changing Acne Mask with Controlled Release. Ingredients. (1) Grapeseed oil 34.28 (2) Ethylenediamine/Hydrogenated Dimer Dilinoleate Copolymer Bis-D1-C14-18 Alkyl Amide 5.0 (3) Dimethicone 2.0 (4) Propyl Paraben 0.3 (5) Jojoba oil 0.5 (6) Sweet Almond oil 4.0 (7) Shea butter 0.2 (8) Mango butter 0.2 (9) Avocado utter 0.2 (10) Murumuru butter 0.2 (11) Color Change Green/Blue dye 0.01 (12) Niacinamide Hydroxybenzoate 5.5 (13) Vitamin E 0.11 (14) Phenoxyethanol 0.7 (15) Zeolite 31.0 (16) Ethylhexylglycerin 0.5 (17) Laureth-3 15.0 (18) Fragrance 0.5. Procedure. Mix (1) to (10) and heat at 70 to 80 C till clear. Cool to 35 to 45 C and all other ingredients and mix. Cool to room temperature. A light green thin paste is obtained. Upon contact with water, it turns blue and releases heat. It is applied topically according to the method of the present invention.

### Example 12

Hair Antiaging Shampoo. Ingredients. (1) Water 64.2 (2) 2-Acetylpyridine N-oxide (1.2) (3) Sodium Lauryl Sulfoacetate 10.0 (4) Disodium Laureth Sulfosuccinate 20.0 (5) Phenoxyethanol 0.7 (6) Chlorphenesin 0.3 (7) PEG-120 Methyl Glucose Dioleate 2.5. (8) Hydrolyzed Soy Protein 0.5 (9) Hydrolyzed Silk Protein 0.5 (10) Oat Extract 0.1. Procedure. Mix (1) to (7) and heat at 60 to 70 C to a clear solution. Cool to 35 to 40 C and add all other ingredients and mix. Cool to room temperature. It is applied topically according to the method of the present invention.

### Example 13

Topical Inflammation Control Massage Lotion. Ingredients percent (1) Water 39.158 (2) Acrylates/C10-30 Alkyl Acrylate Crosspolymer 0.5 (3) Escin 0.1 (4) Sodium Stearyl Phthalamate 1.0 (5) Sodium Hydroxide 0.142 (6) Cetyl Alcohol 4.0 (7) Phenoxyethanol 0.7 (8) Chlorphenesin 0.3 (9) Grapeseed oil 10.0 (10) Ethylhexylglycein 0.5 (11) Polysorbate-20 10.0 (12) PEG-6 2.0 (13) Tetrahydrocurcuminoids 0.1 (14) Magnolol 0.1 (15) Paeonol 0.2 (16) Fragrance 1.0. Procedure. Mix (1) to (11) and heat at 80 to 90 C till clear. Cool to 45 to 55. Pre-mix (12) to (16) and add to main batch and mix. Cool to room temperature and adjust pH to 7.5. It is applied topically according to the method of the present invention.

### Example 14

Anti-inflammatory Make-up Remover Fluid. Ingredients percent (1) Water 39.158 (2) Acrylates/C10-30 Alkyl Acrylate Crosspolymer 0.5 (3) Harpagoside 0.1 (4) Sodium Stearyl Phthalamate 1.0 (5) Sodium Hydroxide 0.142 (6) Cetyl Alcohol 4.0 (7) Phenoxyethanol 0.7 (8) 1,2-Octanediol 0.3 (9) Grapeseed oil 10.0 (10) Methyl Soyate 30.0 (11) Ethylhexylglycein 0.5 (12) Polysorbate-20 10.0 (13) PEG-6 2.0 (14) Tetrahydrocurcuminoids 0.1 (15) Magnolol 0.1 (16) Paeonol 0.2 (17) Fragrance 1.0. Procedure. Mix (1) to (12) and heat at 80 to 90 C till clear. Cool to 45 to 55. Pre-mix (13) to (16) and add to main batch and mix. Add (17) and mix. Cool to room temperature and adjust pH to 7.5. It is applied topically according to the method of the present invention.

### Example 15

### A Method of Treatment for Skin Condition or Disorder

(i) A composition is first prepared according to Examples 1 to 14, which includes a compound having chemical structure in accordance to FIG. 1 and
(ii) Said composition is applied topically at the site of affliction in a sufficient quantity, and
(iii) Said application is repeated to complete the treatment as desired.

### Example 16

### A Method of Treatment for Dark Skin

(i) A composition is first prepared according to Example 5 and 7, which includes a compound having chemical structure in accordance to FIG. 1 and
(ii) Composition of example 7 is first applied at the site of affliction in a sufficient quantity, then rinsed off, and then composition of example 5 is then applied at the said site in a sufficient quantity, and
(iii) Said application is repeated to complete the treatment as desired.

### Example 17

### A Method of Treatment for Enzyme Dysfunction

(i) A composition is first prepared according to Examples 1 to 14, which includes a compound having chemical structure in accordance to FIG. 1 and
(ii) Said composition is applied topically at the site of affliction in a sufficient quantity, and
(iii) Said application is repeated to complete the treatment as desired.

1. A topical method of treatment for dysfunction of dermal enzymes comprising;
(i) An Extra-cellular, Matrix metalloprotease regulating agent, and
(ii) An Intra-cellular Ubiquitin—Proteasome regulating agent, and
(iii) An epidermal melanocyte-regulating agent, and, wherein,
(iv) Said extra-cellular agent, said intracellular agent, and said epidermal agent is a multi-function hydroxyaryl compound in accordance to formula (I): 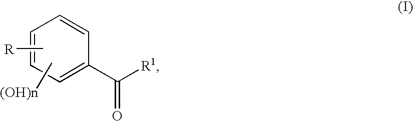
Wherein;
(OH)n is one, two, or three OH substituents, one of which is 2-hydroxy;
Ris one, two, or three substituents each independently selected from the group consisting of H, Alkyl, Cycloalkyl, Aralkyl, Aryl, Cl, Br, NH₂, NH-Alkyl, N(Alkyl)₂, O-Alkyl, S-Alkyl, Heterocyclic, and Heteroaryl; and
R¹ is selected from the group consisting of Methyl, Ethyl, Alkyl, Aralkyl, Heterocyclic, and Heteroaryl; and, wherein,
(v) Said multi-function hydroxyaryl compound is applied topically at a desired site in a sufficient quantity; and, wherein,
(vi) Said application having been done either by a manual or a mechanical method, or a combination thereof; and, wherein,
(vii) Said topical application causes the desired treatment of said dysfunction.

2. A method according to claim 1, wherein said multi-function compound is Resacetophenone.

3. A method according to claim 1, wherein said dysfunction consists of a combination of Matrix Metalloproteases, Ubiquitin—Proteasomes, and epidermal Melanocytes.

4. A method according to claim 1, wherein said multi-function compound is further selected from the group consisting of Resacetophenone, 2-hydroxyacetophenone, 3-hydroxyacetophenone, 4-hydroxyacetophenone, 2,3-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, 2-Acetyl resorcinol, 4-Acetyl resorcinol, 3,4-Dihydroxyacetophenone, Quinacetophenone, 1-(3-Hydroxy-4-methoxy-5-methylphenyl)ethanone, 1-(3-hydroxy-4-methoxyphenyl)ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, 2-hydroxypropiophenone, 3-hydroxypropiophenone, 4-hydroxypropiophenone, 2,3-dihydroxypropiophenone, 2,4-dihydroxypropiophenone, 2,5-dihydroxypropiophenone, 2,6-dihydroxypropiophenone, 3,4-dihydroxypropiophenone, 3,5-dihydroxypropiophenone, 2,4,6-trihydroxypropiophenone, 2,3,4-trihydroxypropiophenone, 2,3,5-trihydroxypropiophenone, 2,3,6-trihydroxypropiophenone, 2,4,5-trihydroxypropiophenone, 3,4,5-trihydroxypropiophenone, phloridzin, phloretin, 1-(2,4-dihydroxyphenyl)-2-hydroxyethanone, (2-hydroxyphenyl)(oxo)acetic acid, 1-(2,6-dihydroxyphenyl)-1-butanone, 1-(1-hydroxy-2-naphthyl)ethanone, 1-(2-hydroxy-1-naphthyl)ethanone, 5,7-dihydroxy-1-indanone, 1-(2-hydroxy-5-methylphenyl)-1,3-butanedione, N-(4-acetyl-3-hydroxyphenyl)acetamide, 4-acetyl-3-hydroxyphenyl acetate, 1,1′-(4,6-Dihydroxy-1,3-phenylene)bisethanone, 1-(1-hydroxy-2-naphthyl)ethanone, 2,3-Dihydro-9,10-dihydroxy-1,4-anthracenedione, 1-hydroxy-2-acetylnaphthalene; 1-hydroxy-2-acetyl-5,6,7,8-tetrahydro-naphthalene; 7-acetyl-8-hydroxyquinoline; 3-acetyl-4-hydroxyacridine; 6-acetyl-7-hydroxybenzothiazole, 2-acetylpyridine, 2-acetylpyrrole, 2-acetylimidazole, 2-acetylthiazole, 2-acetylpyrimidine, 2-acetylindole, 2-acetyl-1-methylpyrrole, 2-acetyl-4-methylpyridine, 1-acetylphenothiazine, 2-hydroxy-1-acetylphenothiazine, 8-hydroxy-9-acetylphenanthrene, 2-acetylpyrazine, 2-acetylquinoline, 2-acetyl-8-hydroxyquinoline, 2-acetyltryptophane, 2-acetyltryptophanamide, 2-acetylpyridine N-oxide, 2-acetylquinazoline, 2-acetylquinoxaline, 3-acetylpyridazine, 6,6′-diacetyl-2,2′-pyridyl, 3-actyl-1,2,4-trizol, and combinations thereof.

5. A method according to claim 1, wherein a carrier base or delivery system is included.

6. A method according to claim 1, wherein said topical method of treatment is for skin condition or disorder caused by dysfunction of said enzymes.

7. A method according to claim 4, wherein said compound is Resacetophenone.

8. A method according to claim 4, wherein said compound is Phloridzin.

9. A method according to claim 5, wherein said carrier base is selected from the group consisting of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and combinations thereof.

10. A method according to claim 5, wherein said delivery system is selected from the group consisting of a traditional water and oil emulsions, suspensions, colloids, microemulsions, clear solutions, suspensions of nanoparticles, emulsions of nanoparticles, anhydrous compositions, and combinations thereof.

11. A method according to claim 6, wherein said skin condition or disorder is further selected from the group consisting of skin aging, skin wrinkles, dark skin, age spots, acne, skin inflammation, loss of cellular antioxidants, loss of collagen, loss of skin pliability, loss of skin suppleness, oily skin, or a combination thereof.

12. A method according to claim 6, wherein said skin condition or disorder is skin wrinkles.

13. A method according to claim 6, wherein said skin condition or disorder is dark skin.

14. A method according to claim 9, wherein said carrier base is a cream.

15. A method according to claim 10, wherein said delivery system is an anhydrous composition.

16. A method according to claim 11, wherein said skin condition or disorder is skin wrinkles.

17. A method according to claim 11, wherein said skin condition or disorder is dark skin.
